(19) United States
(12) Patent Application Publication
(10) Pub. No.: US 2015/0166960 A1
(43) Pub. Date: Jun. 18, 2015

(54) METHOD FOR PRODUCING FUNCTIONAL FUSION TISSUE

(71) Applicant: BRANDENBURGISCHE TECHNISCHE UNIVERSITAET COTTBUS-SENFTENBERG, Senftenberg (DE)

(72) Inventors: Mario Lehmann, Grosskoschen (DE); Frank Martin, Senftenberg (DE); Ursula Anderer, Senftenberg (DE)

(21) Appl. No.: 14/407,505

(22) PCT Filed: Jun. 21, 2013
(86) PCT No.: PCT/EP2013/062991

§ 371 Date: Dec. 12, 2014

(30) Foreign Application Priority Data
Jun. 21, 2012 (EP) 12173056.8

(51) Int. Cl. C12N 5/077; A61F 2/28; A61L 27/38; A61F 2/30

(52) U.S. Cl. CPC C12N 5/0655; A61F 2/30756; A61F 2/28; A61F 2/3094; A61L 27/3895; A61L 27/3817; A61F 2002/30762; A61F 2002/30764; C12N 2513/00; A61L 2430/06; A61L 2430/02

(57) ABSTRACT

The present invention relates to a universal method for producing functional tissue. The invention relates to the method for producing functional fusion tissue, the functional fusion tissue obtainable by this method, and use thereof in particular as a pharmaceutical preparation, drug, transplant, implant, food and test system. The invention in particular relates to the production of functional cartilage tissue and bone tissue from articular cartilage and bone.

The present invention relates to a universal method for producing functional fusion tissue. The invention also relates to the functional fusion tissue obtainable by this method, and to the use thereof, in particular as a pharmaceutical preparation, drug, transplant, implant, food and test system. The invention in particular relates to the production of functional cartilage tissue and bone tissue from articular cartilage and bone.

Although articular cartilage demonstrates notable resilience, this tissue is unable or is hardly able to repair itself, and untreated legions may lead to osteoarthritis. This low potential for spontaneous regeneration has led to the development of cell therapies, such as autologous chondrocyte implantation (ACI), with the aim of providing a functional and pain-free repair of articular cartilage defects. Techniques of this type, however, cannot guarantee cartilage regeneration, and there have not previously been any adequate long-term studies. Consequently, there is a high need for methods for cartilage regeneration, for example in young active patients with traumatic legions or even with systems of cartilage degeneration.

Numerous studies have been carried out with chondrocytes which have been isolated from cartilage of cattle, rabbits or sheep. The obtained data and animal-based concepts of this type, however, cannot be transferred to the human situation. Detailed biochemical and molecular studies with human chondrocytes have been hindered by a series of factors, such as the lack of availability of human tissue in conjunction with the very small number of cells available in a biopsy, the limited proliferation capacity and the high phenotypic instability of cultivated chondrocytes.

A chondrocyte (cartilage cell) is a cell that originates from chondroblasts and that is established in the cartilage tissue. Together with the intercellular substances (extra cellular matrix (ECM)), the chondrocytes form the primary components of cartilage.

It is already known, for example by mimicry of certain processes of embryonal development, for example to cultivate human cartilage cells three-dimensionally on an agarose substrate, such that cell aggregates are produced which are superior to the monolayer cells in terms of their differentiation ability and which for example have cartilage-like properties. These properties, which reflect the native articular cartilage tissue as accurately as possible, are characterised by the expression of collagen II (primary structural protein in the extracellular matrix of hyaline cartilage), of proteoglycans, for example aggrecan, and the intracellular chondrocyte-specific protein S100. In addition, it is desirable for the expression of collagen I, which is necessarily up-regulated during the cell multiplication phase in the monolayer culture, to be reduced again in the cell aggregates, since this protein is practically absent in native articular cartilage. The cell aggregates thus generated (spheroids) have been offered since 2002 for example by the company co.don in order to therefore treat primarily smaller cartilage defects, caused by injury, in younger patients. To this end, natural articular cartilage tissue is removed from the patient and the cells isolated therefrom are transplanted as spheroids following multiplication and 3D culture (Chondrospheres®). However, the cell aggregates thus produced are limited in terms of their differentiation status and often demonstrate only a relatively weak local expression of collagen II, which is important, and also only marginal synthesis of proteoglycans, but with relatively strong expression of collagen I, which is undesirable. In order to further improve the differentiation of these cell aggregates, there is the possibility to enrich the culture medium with certain bioactive substances. These include primarily the growth factors TGF-β1-3 and also L-ascorbic acid (vitamin C), which are described in many instances in the literature, as cofactor for the collagen synthesis. As a result, the proteoglycan and collagen II synthesis in particular can be intensified in the cell aggregates that are cultivated in the presence of these factors. However, this biochemical stimulation on the one hand is often insufficient to induce the differentiation of the cartilage cells in the 3D cell aggregate to an extent that cartilage-typical constructs are produced, and on the other hand the use of growth factors such as TGF-β is disputed in particular for clinical use in humans. Proteins from the TGF-β family regulate a large number of cellular processes, such as proliferation, differentiation, growth, migration, etc., but may also be involved pathologically in tumour development or may promote the growth of existing tumours and favour the metastasis thereof.

Since, without a given framework structure, the self-organisation of a tissue is usually absent, the use of frameworks (scaffolds) is often key, for example in tissue engineering (TE) of blood vessels. Here, the selection of a suitable framework is difficult in practice and often impossible.

Vinatier et al. (Current Stem Cell Research & Therapy (2009), LNK-PUBMED: 19804369,vol. 4, no. 4, p. 318-329) gives an overview of the factors and framework materials that are used in the tissue engineering of cartilage.

Fleming et al. (Developmental Dynamics: An official publication of the American Association of Anatomists (2010) LNK-PUBMED: 19918756, vol. 239, no. 2, p. 398-406) discloses the production of artificial blood vessels, wherein two vascular spheroids, which each have a central lumen, are fused to form a spheroid of larger diameter and having a larger central lumen.

Anderer et. al (International Journal of Artificial Organs (2002), Milan, IT, vol. 25, no. 7, p. 675) describes a method for the in vitro production or autologous fusion tissue from cartilage, but does not mention the fusion of spheroids. The tissue thus produced differs from native tissue significantly in view of the expression of extracellular matrix proteins which are necessary for functional cartilage tissue.

The cultivation of human cells in the form of three-dimensional cell aggregates, for example in the form of what are known spheroids, is already authorised for clinical use in humans, for example as autologous cartilage transplant (DE 100 13 223). DE 100 13 223 concerns a method for the in vitro production of three-dimensional cartilage tissue and bone tissue from bone stem cells, cartilage stem cells or mesenchymal stem cells. Here, the cells are firstly cultivated in a monolayer culture and are then cultivated in suspension until a cell aggregate is produced that contains at least 40 vol % extracellular matrix, in which differentiated cells are embedded. In the method, cell aggregates are produced by cultivating cells for at least 1-2 weeks in cell culture vessels coated with agarose. DE 100 13 223 also discloses the fact that, depending on the desired tissue size, at least two of the produced cell aggregates can be fused, but does not specify specific conditions for fusion. However, the tissues produced by this method differ considerably in terms of their functionality and expression patterns, in particular in view of the expression of collagen type II, from the corresponding native tissues.

U.S. Pat. No. 7,887,843 B2 discloses a method for the in vitro production of three-dimensional cartilage tissue and bone tissue, wherein spheroids are produced from bone stem cells, cartilage stem cells or mesenchymal stem cells by cultivating 1×10⁵ cells for at least two weeks. The spheroids produced in accordance with U.S. Pat. No. 7,887,843 B2 have a diameter of 500-700 μm after one week. Without the method step of fusion of spheroids, fusion tissue having a content of extracellular matrix (ECM) of 90% is obtained from this cell culture after 3 months. U.S. Pat. No. 7,887,843 B2 mentions the possibility of fusing two or more spheroids in a second step in order to produce larger tissue pieces, but does not specify specific conditions for this. Anderer et al. (Journal of Bone and Mineral Research (2002), New York, N.Y., US, vol. 17, no. 8, p. 1420-1429) also discloses a method for the in vitro production of three-dimensional cartilage tissue. In accordance with this method, 1×10⁵ or 2×10⁵ chondrocytes are cultivated for 5 days, 2 weeks 1, 2 and 3 months in order to produce spheroids. The nutrient supply within the spheroids is ensured by using only spheroids less than 800 μm in size, wherein 2-10 spheroids can be coalesced in a second step. The spheroids used for this purpose have a diameter of 350-500 μm. Anderer et al. also presents the fusion of three spheroids that are 16 days old.

The cartilage tissues produced in vitro described in the prior art do not present any expression patterns of essential matrix proteins, such as collagen type II, that are similar to those of native tissues. Rather, the composition of the extracellular matrix (ECM) produced in vitro from the chondrocytes deviates significantly from native cartilage tissue. However, the composition of the ECM is key for the biological functionality of the cartilage, for example the mechanical load-bearing capacity. There is therefore a need for new techniques for producing functional fusion tissue, in particular functional cartilage tissue.

The invention provides techniques and means with which functional tissue can be produced. The invention relates to functional fusion tissues that are produced by a novel method and that correspond or largely correspond in terms of the biological functionality thereof to native tissue. By way of example, a very high degree of similarity in view of the expression of collagen type II and specific proteoglycans is found when cartilage tissue produced in vitro in accordance with the method according to the invention is compared with native cartilage tissue. The method according to the invention is characterised in that the in vitro cultivation is carried out in a particular three-dimensional environment (3D environment). The three-dimensional environment according to the invention is achieved by the size and number of spheroids used. This three-dimensional environment causes the spheroids to fuse spontaneously and independently. With the method according to the invention, larger fusion tissues can be produced in a shorter time compared with the methods described in the prior art, which surprisingly has a particularly advantageous effect on the biological functionality of the tissue produced.

The invention relates to a method for producing functional fusion tissue, characterised in that spheroids are produced and spheroids having a diameter of at least 800 μm, preferably having a diameter or 800-1400 μm, are selected, wherein at least 5 spheroids having a diameter of at least 800 μm, preferably having a diameter of 800-1400 μm, are fused.

In the method according to the invention, cells from tissue of human or animal origin are preferably used that are isolated from the tissue and from which the spheroids are produced. In one embodiment of the invention, spheroids are produced by isolating cells from their normal environment and cultivating, that is to say multiplying, said cells.

The cells generally dedifferentiate wholly are partially due to the isolation of the cells from the tissue. The method according to the invention is suitable for producing functional fusion tissue from dedifferentiated cells. One embodiment of the method therefore concerns the use of dedifferentiated cells for the production of spheroids.

Alternatively however, it may also be that dedifferentiated cells are not used or are only used in part for the production of spheroids.

The invention relates to a method for producing functional fusion tissue, characterised in that,

a) cells are isolated from tissue of human or animal origin,

b) the isolated cells are introduced into another environment and are multiplied,

c) spheroids are produced from the multiplied cells,

d) five or more spheroids having a diameter of 800 μm, preferably of 800-1400 μm, are fused.

The invention also relates to a method for producing functional fusion tissue, characterised in that

a) cells are isolated from tissue of human or animal origin,

b) dedifferentiated cells are produced from the isolated cells,

c) spheroids are produced from the dedifferentiated cells,

d) five or more spheroids having a diameter of 800 μm, preferably of 800-1400 μm, are fused.

In one embodiment, spheroids are produced which have different diameters, wherein in a second step those spheroids having a diameter of at least 800 μm, preferably of 800-1400 μm, are selected from the produced spheroids.

In another embodiment, spheroids having a diameter of at least 800 μm, preferably of 800-1400 μm, are produced directly. Spheroids of this size can be obtained for example by sowing 3×10⁵ to 5×10⁵ cells per well of a microtiter plate (96-well plate), preferably 4×10⁵ cells per well, particularly preferably 3×10⁵ cells per well. These cells can be cultivated for 1 to 3 days, preferably 36 to 60 hours, particularly preferably 2 days, in order to produce spheroids with a size of at least 800 μm, preferably of 800-1400 μm diameter.

Spheroids having a diameter of 800 μm, 900 μm, 1000 μm, 1100 μm, 1200 μm, 1300 μm, 1400 μm or more are particularly suitable for the method.

The invention relates to a method with which functional fusion tissue, also referred to as “fusion culture system”, “fusion culture” and “functional tissue”, can be produced without further aids, such as frameworks. The functional fusion tissue produced with this method consists here of fusions, that is to say a plurality of spheroids fused to one another. The functional fusion tissue produced with the aid of the method according to the invention is preferably present in the form of microtissue.

“Functional fusion tissue” means that the tissue corresponds largely to native tissue or is identical to native tissue. In the sense of this invention, the functional fusion tissue corresponds largely to natural tissue, for example in respect of the expression or expression patterns of the extracellular matrix, and for example in respect of the expression or expression patterns of structural macromolecules such as collagen, in particular collagen type II and/or collagen type I and/or S100 protein and/or the tissue-specific proteoglycans, for example cartilage-specific proteoglycans (for example articular cartilage tissue in the case of functional cartilage tissue). The expression or expression patterns can be detected histologically or immunohistologically, for example. In the case of functional fusion tissue of other tissue types, the detection can be implemented for example via specific surface proteins or components of the extracellular matrix, of which the expression pattern in the functional fusion tissue corresponds or largely corresponds to that of the natural tissue in question.

Within the scope of the present invention, cells that are closely related in terms of function and that can form a tissue or are part of a tissue are used for the production of functional fusion tissue, for example fibroblasts, hepatocytes, nerve cells, osteoblasts, osteoclasts, and keratinocytes. The tissue from which the cells are isolated is selected for example from musculoskeletal tissue, skeletal tissue, cartilage, bone, meniscus, epithelial tissue, connective tissue, supporting tissue, muscular tissue, smooth muscle, heart muscle, nerve tissue, functional tissue (parenchyma), intermediate tissue (interstitium), organ tissue, for example of the liver, kidney, adrenal cortex, stomach, pancreas, heart, lung, skin, cornea, subcutaneous tissue, intestinal tract, bone marrow, brain, thyroid, spleen, joint, or tendon. The functional fusion tissue produced by the method according to the invention is similar to the native tissue in terms of the composition and expression of one or more components, preferably of at least two components. Functional fusion tissue from chondrocytes for example corresponds to the native cartilage tissue in terms of the expression of collagen type I and type II, S100 protein and/or tissue-specific proteoglycans.

Native articular cartilage tissue has a composition of the extracellular matrix formed of approximately 60 to 80% water in relation to the wet weight of the articular tissue. The high water content is important for the mechanical load-bearing ability of the cartilage tissue and, together with the proteoglycans, is important for the “sponge effect”. Besides water, natural articular cartilage contains the structural macromolecules of the matrix, such as collagens, proteoglycans and non-collagen proteins. Here, the structural macromolecules account for approximately 20 to 40% of the wet weight of the articular cartilage tissue.

The functional fusion tissue produced by the method according to the invention from human chondrocytes for example has a water content from 50 to 90%, preferably 55 to 85% or 60 to 80%, particularly preferably 65 to 75% or 70% in relation to the wet weight of the functional fusion tissue. The functional fusion tissue for example has 10 to 50% structural macromolecules of the matrix, preferably 15 to 45% or 20 to 40%, particularly preferably 25 to 35% or 30% in relation to the wet weight of the functional tissue. Here, the respective water contents, structural macromolecules and optionally further components add up to 100%.

In the case of native articular cartilage tissue, the collagen proportion is 60%, the proteoglycan proportion is 25 to 35%, and the non-collagen protein and glycoprotein proportion is 15 to 20% in relation to the cartilage dry weight. Here, the primary collagen is collagen type II. Collagen type II accounts for approximately 90 to 95% of the total collagen content of the native articular cartilage tissue.

Functional fusion tissue produced by the method according to the invention from human chondrocytes for example contains 50 to 70%, preferably 55 to 65%, particularly preferably 60% collagen in relation to the dry weight of the functional fusion tissue.

Functional fusion tissue produced by the method according to the invention from human chondrocytes for example contains 15 to 45%, preferably 20 to 40%, particularly preferably 25 to 35% proteoglycans in relation to the dry weight of the functional fusion tissue. The proteoglycan content of the fusion tissue produced by the method according to the invention from human chondrocytes for example is 150 to 550 μg, preferably 170 to 500 μg or 200 to 460 μg proteoglycans per mg fusion tissue, determined quantitatively

Functional fusion tissue produced by the method according to the invention from human chondrocytes contains collagen type II as one of the most important components of the extracellular matrix in the articular cartilage tissue and is expressed in at least 70 to 99%, preferably 75 to 97%, particularly preferably in 80 to 95% of the tissue section of fusions. The percentages are given following computer-assisted evaluation or calculation of the positive immunofluorescence for the protein collagen type II in relation to the total tissue section of the fusion tissue.

Functional fusion tissue produced by the method according to the invention from human chondrocytes for example contains 80 to 99% or more, preferably 85 to 98% or 80 to 98%, particularly preferably 85 to 97% or 90 to 96% or 95% collagen type II in relation to the total collagen content of the functional fusion tissue.

Native human articular cartilage tissue contains approximately 1 to 5% cartilage cells (chondrocytes) in relation to the tissue volume. Here, the cartilage cells are the essential producers of the matrix molecules of the functional native tissue.

Functional fusion tissue produced by the method according to the invention from human chondrocytes for example contains 0.1 to 10% or 0.2 to 8%, preferably 0.4 to 6% or 0.5 to 5%, particularly preferably 0.7 to 3% or 0.9 to 1%, chondrocytes, which produce the extracellular matrix molecules. The functional cartilage tissue thus contains at least 70 to 90%, preferably at least 80% ECM.

One embodiment of the invention concerns a method according to the invention, characterised in that 5 to 10 or more, preferably 6 or 7, particularly preferably 8 or 9 spheroids are fused. In a particularly preferred embodiment of the method, 5 spheroids are fused in order to produce the functional fusion tissue.

Functional fusion tissue of different size can be produced with the method. For example, individual spheroids have a diameter of approximately 750-1500 μm, preferably of 800-1400 μm, particularly preferably of 1000-1300 μm. Accordingly, the number of fused spheroids also determines the size of the functional fusion tissue. The size of the functional fusion tissue, besides being dependent on the number of fused individual spheroids, is also dependent on the self-arrangement (form) of said spheroids. In the preferred case of 5 individual spheroids in the fusion, the dimensions could be specified for example by 2000-3000 μm×3000-4000 μm. In the case of more than 5 individual spheroids, the dimensions are larger accordingly. The size can be determined histologically, for example. The corresponding methods are known to a person skilled in the art.

Here, the special arrangement of the dedifferentiated cells at the surface of individual spheroids is key as well as the presence of at least one differentiation inducer for production of functional fusion tissue. The close spatial arrangement of 5 or more spheroids constitutes a differentiation inducer. The closeness of a number of spheroids to one another leads here not only to the coalescence (fusion) of a number of smaller cell aggregates (spheroids), but also induces the redifferentiation of the dedifferentiated cells in the spheroids and the fusion tissue formed therefrom. Therefore, not only are larger microtissues produced with the method according to the invention, but also microtissues that contain redifferentiated cells and correspond entirely or largely in terms of their biological functionality to the native tissue.

A feature essential for the invention is therefore the particular 3D environment (also referred to as particular 3D culture) of the spheroids during production of the functional fusion tissue. The term “3D environment” means that an individual cell can come into contact with another cell in any direction. The spherical spheroids are contacted in a small space in a particular “3D environment”. Fusion tissue is formed by the contact, thus possible, between the cells in the edge region of the individual spheroids with the cells in the edge region of the other spheroids, that is to say larger tissue pieces can be produced. The particular 3D environment is preferably produced in accordance with the invention by bringing the spheroids spontaneously and independently into a position in which they can fuse in an ideal manner. The ideal position is characterised for example in that cell-cell contacts (tight junctions, desmosomes and the like) can form and the extracellular matrix formation is induced, wherein the formed ECM has the organotypic properties in view of the expression pattern. The particular 3D environment is then formed when 5 or more spheroids having a diameter of at least 800 μm are fused. The formation of functional fusion tissue is induced by fusion of 5 or more spheroids having a diameter of at least 800 μm.

In one embodiment, the individual spheroids, preferably 5 individual spheroids, having a diameter of at least 800 μm are applied to a concave cultivation surface, so that these assume the ideal position, that is to say closeness, to one another independently (spontaneously) when applied to the surface so as to be able to fuse. In one embodiment of the invention, the concave surface is provided by a method comprising the following steps:

a) agarose is dissolved in the cell culture medium and b) a defined volume, for example 100 μl, is poured as hot as possible into a culture vessel, for example into a well of a 96-well plate. The agarose becomes solid once cooled. This occurs at staggered intervals from the edges towards the centre of the cultivation dish, whereby the agarose surface “draws up” at the sides of the well and on the whole forms an ideal concave surface. Other suitable concave surfaces can be produced accordingly.

In one embodiment of the invention, the individual spheroids, preferably 5 individual spheroids, having a diameter of at least 800 μm are applied to a cultivation surface that is structured such that the spheroids preferably independently (spontaneously) assume the ideal position, that is to say closeness, to one another so as to be able to fuse. Here, the cultivation surface is structured such that the spheroids preferably do not interact with the cultivation surface, but preferably interact with one another. An accordingly structured, suitable cultivation surface is, for example, a hydrophobic surface such as agarose.

The method according to the invention is preferably carried out in vitro in order to produce microtissue (functional fusion tissue) in vitro. This can be used for example as transplant, implant, preparation, for example tissue preparation, drug or food or for test systems.

The invention therefore relates to a method for producing functional fusion tissue with use of a special culture system together with factors that promote the differentiation, but without the additional aid of framework material. The method comprises two aggregation steps: in the first aggregation step individual isolated spheroids are produced from the isolated cells, which may be dedifferentiated partially or completely, and said spheroids are then further fused in the second aggregation step of the fusion cultivation in the presence of components that are relevant for cell condensation and cell communication (referred to as “differentiation inducer” within the scope of this invention.

In accordance with the invention, a number of layers of the surfaces of in vitro tissues, in particular of spheroids, are arranged in close contact with one another (“cultivation in 3D environment” in accordance with the invention) in the second aggregation step, and the mesenchymal condensation is thus imitated. Here, the differentiation inducer is the cell-cell interaction and/or cell-matrix interaction and/or the formation of gap junctions.

Both the combination of a plurality of individual spheroids with already fused tissue or the combination of a plurality of individual spheroids are included in accordance with the invention.

A further embodiment of the invention concerns a method according to the invention, characterised in that at least 1×10⁵ cells/well of a microtiter plate, preferably 3×10⁵ cells/well, particularly preferably 5×10⁵ cells/well or more are fused.

In the sense of this method, “differentiation inducers” are preferably mechanical and/or chemical and/biochemical differentiation inducers. By way of example, the cultivation in 3D environment with at least 5 spheroids, the cultivation in the presence of mechanical stimuli, such as pressure application, cultivation in bioreactors such as a rotating wall vessel or spinner flasks are suitable as mechanical differentiation inducers. For example, ascorbic acid, in particular L-ascorbic acid and derivatives of ascorbic acid, such as ascorbate-2-phosphate, are suitable as chemical differentiation inducers. For example, proteins such as proteins of the TGF-β superfamily, for example the TGF-β isoforms (TGF-β1, TGF-β2, TGF-β3) and bone morphogenetic proteins (BMP-2, BMP-3, BMP-4, BMP-5, BMP-6, BMP-7), growth differentiation factor, such as GDF-5 and GDF-10, and insulin-like growth factor such as IGF-1 are suitable as biochemical differentiation inducers. In principle, all differentiation-modulating substances are suitable as differentiation inducers, for example also glucocorticoids such as dexamethasone. Differentiation inducers in the sense of the present invention are all components and factors that are relevant for cell condensation and cell communication or that influence cell condensation and cell communication. One or more differentiation inducers can be used in the method in accordance with the invention. The usability of individual differentiation inducers and also the suitability of combinations of a number of differentiation inducers can be checked by a person skilled in the art, for example on the basis of the expression patterns of collagen type II, tissue-specific proteoglycans, collagen type I and/or S100 protein.

A preferred embodiment of the invention concerns a method according to the invention, characterised in that the further differentiation inducer(s) is/are selected from the differentiation inducers TGF-β together with ascorbic acid, in particular TGF-β2 together with L-ascorbic acid.

A further embodiment of the invention concerns a method according to the invention, characterised in that the solid tissue from which the cells are isolated is of human or animal origin. The solid tissue may be of entodermal, ectodermal and/or mesodermal origin. Mesenchymal stem cells (MSCs) have a high proliferation and differentiation potential. Adult mesenchymal stem cells contribute to the maintenance and regeneration of supporting and connective tissue, such as bone, cartilage, muscle, ligaments, tendons and fatty tissue. In addition, they promote growth and development of the precursor cells of the blood in bone marrow. MSCs from different tissues (bone marrow, cartilage, fatty tissue, muscle, liver tissue, blood, amniotic fluid) can be cultivated and differentiated in vitro in different tissue.

The method for producing functional fusion tissue makes it possible to combine individual cell aggregates to form a larger “cartilage-like” tissue, which then also has significantly improved properties in terms of differentiation and tissue quality, which ultimately favours a use for example as an in vitro test system for the pharmaceutical industry or as a transplant in regenerative medicine.

Within the scope of the method according to the invention, animal cells, in particular human cells, are preferably used. The cells are preferably adult cells. The cells, for example, are cells locked in the cell cycle. Freshly isolated cells are preferably used. A further embodiment of the invention concerns a method according to the invention, characterised in that the cells are freshly isolated and/or are post-mitotic animal or human cells. In a preferred embodiment of the invention, adult human chondrocytes, in particular freshly isolated post-mitotic chondrocytes (cartilage cells) are used. In a further preferred embodiment of the invention, adult human bone cells, in particular osteocytes and/or osteoblasts, are used.

A further embodiment of the invention concerns a method according to the invention, characterised in that a plurality of different cells are used in order to produce complex functional tissue. For example, cells that comprise chondrocytes can be used.

A further embodiment of the invention concerns a method according to the invention, characterised in that the dedifferentiated cells are produced by culture in monolayer.

The cultivation of the isolated cells, for example in method step b), is used to multiply the isolated cells. To this end, the cells are isolated from the tissue and are introduced into a new environment, in which they multiply ideally. Corresponding methods are known to a person skilled in the art. The introduction of isolated cells into the new environment results in the dedifferentiation of the isolated cells. Due to the dedifferentiation, the functionality of the cells changes, which can be detected for example by the modified expression patterns of collagen type II, collagen type I, tissue-specific proteoglycans and/or S100 protein. One embodiment of the invention concerns a method according to the invention, wherein the isolated cells are multiplied by being cultivated in monolayer culture, for example for two or more passages.

The invention relates to a method for producing functional fusion tissue comprising the following steps

a) expansion of isolated cells, in particular of chondrocytes, in a monolayer culture with dedifferentiation of the cells,

b) aggregation of the isolated and dedifferentiated cells to form spheroids,

c) aggregation of the spheroids with redifferentiation of the cells by cultivation in a 3D environment (fusion cultivation) optionally in the presence of further differentiation inducers, such as TGF-β2 and/or L-ascorbic acid.

A further embodiment of the invention concerns a method according to the invention, characterised in that the method comprises a further step, in which the formation of spheroids is stimulated. To this end, the isolated or multiplied or dedifferentiated cells are introduced into an environment in which the cells preferably attach to other cells and not to the surface of the environment. To this end, vessels having a hydrophobic surface for example can be used for the cultivation. A further embodiment of the invention therefore concerns a method, wherein dedifferentiated cells are cultivated on a hydrophobic surface, preferably on an agarose layer, following the dedifferentiation. The cultivation on agarose stimulates spheroid formation.

A further embodiment of the invention concerns a method according to the invention, characterised in that the dedifferentiated cells are cultivated, following dedifferentiation, for 1 to 5 days, preferably two days, on agarose or another hydrophobic surface.

The dedifferentiation and redifferentiation of chondrocytes can be detected via S100, collagen type II, collagen type I and tissue-specific proteoglycans. The dedifferentiation of the cells is characterised in that S100, collagen Type II, and tissue-specific proteoglycans are expressed to a reduced extent and collagen type I is expressed to an increased extent. The differentiation of native cells and the differentiation of functional fusion tissue are characterised by a high expression of collagen type II, tissue-specific proteoglycans, S100 protein and by a low expression of collagen I.

For the cultivation, conventional culture mediums are used in accordance with the invention, preferably without addition of antibiotics or fungistatics. Serum is preferably also added to the culture medium, for example human serum in a concentration of approximately 1% to 20%, preferably 5 to 10%. In accordance with the invention, all conventional culture media are suitable, for example HAMS, alpha medium, DMEM, MEM. A particular embodiment of the method according to the invention concerns the use of alpha medium and HAMS F12 (1:1) as culture medium, wherein L-glutamine and human serum are preferably added.

The fusion of the spheroids takes place in the method according to the invention for example for 3 to 7 weeks, preferably 4 or 5 weeks, particularly preferably 6 weeks. The fusion, for example according to step d) of the method, is carried out in a particular embodiment with 5 spheroids/well of a microtiter plate and preferably in the presence of TGF-β2 and optionally L-ascorbic acid.

The invention also relates to a functional fusion tissue obtainable by a method according to the invention.

One embodiment of the invention concerns functional human cartilage tissue, which is obtained when human chondrocytes are used as cells in the method according to the invention. The functional cartilage tissue is characterised in that the collagen proportion is 50 to 70% and the proteoglycan proportion is 15 to 45% in relation to the dry weight of the functional cartilage tissue. The functional cartilage tissue is also characterised in that the proportion of collagen type II is 85 to 98% in relation to the total collagen content of the functional cartilage tissue. In another embodiment of the method, collagen type II is expressed in at least 80-95% of the tissue sections of fusions, in relation to the total tissue section of the functional human cartilage tissue.

The invention, for example, comprises functional musculoskeletal tissue, functional skeletal tissue, functional cartilage tissue, functional bone tissue, functional meniscus tissue, functional epithelial tissue, functional connective tissue, functional supporting tissue, functional muscular tissue, functional smooth muscle, functional heart muscle, functional nerve tissue, functional function tissue (parenchyma), functional intermediate tissue (interstitium), functional organ tissue, for example functional liver tissue, functional kidney tissue, functional adrenal cortex, functional stomach tissue, functional pancreas tissue, functional heart tissue, functional lung tissue, functional skin tissue, functional cornea tissue, functional subcutaneous tissue, functional tissue of the intestinal tract, functional bone marrow, functional tissue of the brain, functional thyroid tissue, functional spleen tissue, functional articular tissue, and functional tendon tissue, which is obtainable by the method according to the invention.

By way of example, the invention comprises functional fusion tissue produced by the method that is an autologous, xenogeneic, allogeneic or syngeneic fusion tissue in view of the donors and recipients in question (human or animal) of the tissue or cells used for the method. In particular, the invention comprises autologous, xenogeneic, allogeneic or syngeneic functional cartilage tissue and bone tissue.

The fusion tissues obtained by the method according to the invention are not only larger compared with those in the prior art, in particular as described in DE 100 13 223, but also differ in terms of their functionality. The fusion tissues according to the invention are constructed from redifferentiated cells and, in terms of function and structure, are similar or very similar or identical to the native tissues from which the used cells were isolated.

In contrast to the tissue produced in U.S. Pat. No. 7,887,843 B2, more cells are cultivated in the method according to the invention over a shorter period of time in order to produce spheroids. The spheroids thus obtained are larger and surprisingly similar to the native tissues in terms of functionality. The fusion tissue produced by the method according to the invention from chondrocytes consists of 0.1 to 10% ECM-producing cells and thus of 90 to 99.9% ECM. The fusion tissues produced by the method described in U.S. Pat. No. 7,887.843 B2 differ from the fusion tissues according to the invention in terms of functionality and the expression patterns of collagen type I, collagen type II and tissue-specific proteoglycans.

The invention also relates to the use of functional fusion tissue obtainable by the method according to the invention, for example as an implant, transplant, functional replacement tissue, or in vitro tissue cultivation, for production of larger transplantable tissues, for production of in vitro and in vivo tissues, and for tissue engineering. In particular, the invention concerns the use or specific application of the fusion tissue according to the invention as autologous, xenogeneic, allogeneic or syngeneic implant, transplant or tissue preparation.

The invention relates to an implant, transplant and functional replacement tissue obtainable by the method according to the invention. The implant, transplant or functional replacement tissue produced in vitro or in vivo can be introduced into the surrounding native tissue of the recipient. No cell division takes place in the functional fusion tissue produced by the method according to the invention, that is to say also in the implant, transplant or functional replacement tissue. If the functional tissue is introduced into native tissue, the cells of the functional fusion tissue then attach to the native tissue (adhesion) and migrate into the gaps (migration), but without dividing. An optimal supply/restoration is thus ensured, but without the risk of an uncontrolled proliferation of the functional replacement tissue.

Alternatively, the at least 5 spheroids having a size of at least 800 μm can be brought to the point where the implant, transplant or functional replacement tissue is to be localised in the recipient, and the fusion can be carried out in vivo. Ideally, a corresponding concave point at the recipient point is prepared for this purpose.

The invention also relates to the use/specific application of the functional fusion tissue according to the invention together with spheroids.

The invention also relates to a preparation, in particular a pharmaceutical preparation or a tissue preparation, a drug, a transplant or an implant consisting of or containing functional fusion tissue, which is obtainable by the method according to the invention and optionally further excipients and additives.

The invention also relates to pharmaceutical preparations, tissue preparations and drugs, in particular suspensions and solutions, in particular injection solutions, which contain the functional fusion tissue according to the invention and optionally further excipients and additives.

The invention also relates to the specific therapeutic/pharmaceutical use of a pharmaceutical preparation according to the invention, a tissue preparation, a drug according to the invention, a transplant according to the invention or implant according to the invention for treating cartilage defects and/or bone defects, in particular traumatic cartilage defects and/or bone defects, lesions, in particular traumatic lesions, cartilage degeneration, bone degeneration, osteoarthritis, and for therapeutic cartilage regeneration and/or bone regeneration in vivo.

The invention also relates to a method for the in vitro production of foods comprising the fusion of at least 5 spheroids having a diameter of at least 800 μm. The invention also relates to the food obtainable thereby.

The invention also relates to the use of the fusion tissue according to the invention for the testing of active ingredients, for example in order to screen new active ingredients, to improve or to validate known active ingredients or in order to develop new indications and fields of application for a known active ingredient or within the scope of pre-clinical and clinical testing for data generation and testing of active ingredients or for testing of harmful substances.

The invention relates to a test system comprising functional fusion tissue and optionally further additives and/or auxiliaries. The invention relates to a test system, for example a test kit, comprising

a) functional fusion tissue, a corresponding preparation, a corresponding drug, a corresponding transplant or implant,

b) optionally further additives and auxiliaries, and

c) detection means.

The invention, for example, also relates to an in vitro test system for experimental pharmacology. Such a test system can be used to identify new active ingredients and to test new and known active ingredients, for example the effect of a drug on bone and/or cartilage. Furthermore, the effect of substances, of synthetic or natural origin, for example on bone and/or cartilage, can be tested, for example foods, natural substances, solvents, polymers, etc. Such a test system can therefore deliver, for example, information concerning the toxicity and possible side effects and can also be used to determine limit values and synergistic effects of substances.

The invention therefore also relates to a method for testing substances to be examined, said method comprising the following steps

a) Producing functional fusion tissue or a test system comprising functional fusion tissue;

b) contacting a substance to be tested with the functional fusion tissues from a),

c) determining/detecting the effect of the substance to be examined on the functional fusion tissue.

The invention also relates to a method for testing substances to be examined, said method being characterised in that

a) functional fusion tissue, a corresponding preparation, a corresponding drug, a corresponding transplant or implant,

b) is brought into contact with one or more substances to be examined,

c) the effect of the substance(s) to be tested on the functional fusion tissue, the preparation, the drug, the transplant or implant is detected.

The use of fusion cultivation methods for the selective, intensified stimulation of the differentiation of human cells of a wide range of origin, particularly cells of all musculoskeletal tissue (not only cartilage cells) is not known in the prior art. In DE 100 13 223 and others and in Anderer and Libera, 2002, the combination of two individual spheroids to form a larger aggregate is disclosed, but not the aggregation of preferably 5 or more spheroids having a size of at least 800 μm (particular 3D environment of the fusion cultivation method according to the invention) with the objective of thus achieving the redifferentiation of the cells, which involves an increased production of cartilage-specific extracellular matrix components, such as collagen II and proteoglycans. Rather, the aggregation disclosed in the prior art was used to enable the generation of a larger cell aggregate.

The present invention for producing tissues by coalescence (fusion) of at least 5 smaller cell aggregates provides selectively larger, clinically applicable tissue, of which the structure and characteristic protein configuration is very similar to the human original tissue. The special arrangement of the cells at the surface of individual spheroids and the resultant cell-cell contact in the event of contact with adjacent spheroids are used here as new differentiation inducers (amplifiers). Here, a key claim of this novel method for in vitro tissue cultivation lies in the provision of larger transplantable tissues, which mimic the respective natural body tissue (for example articular cartilage). This method can be transferred to all other tissues and can therefore be used universally.

The present invention makes it possible to line and thus regenerate cartilage defects of various size, for example in the knee joint, with a smaller number of larger, better differentiated and functional in vitro tissue. A further key aspect of the functional fusion tissue according to the invention is that, with the method of fusion culture according to the invention, a possibility has been found for producing considerable cartilage properties of the native tissue in the functional in vitro tissues, without the use of differentiation-promoting factors, such as TGF-β. For conformation, the fusion culture was combined with biochemical stimuli in order to demonstrate a synergistic effect with regard to cartilage-specific differentiation. The fusion culture effectively induces in the basal medium the differentiation of the cartilage cells in the functional fusion tissue without any addition of growth factors, ascorbic acid or the use of synthetic framework matrices and is therefore significantly superior to individual cell aggregates (individual spheroids). The method according to the invention enables the production of a new generation of transplantable tissue for therapeutic application with and without additional growth factors.

Another possible application concerns the use of the in vitro produced functional fusion tissue as a base for a platform technology for testing substances, for example in the pharmaceutical industry, the chemical industry, or the food industry. By way of example, a test system could therefore be provided in the field of rheumatic diseases in order to analyse the physiology of cartilage cells in healthy and arthrotically induced tissue imitations, in particular also in view of the response to allegedly therapeutic substances.

The generation of body-like tissue by the fusion of individual cell aggregates is possible in vitro with the method according to the invention. It has been possible to demonstrate that the isolated chondrocytes from various patients following the dedifferentiation phase in the monolayer redifferentiate again merely by the fusion culture without the addition of bioactive stimulants. A solution approach for the common problem of donor dependency is thus also created, such that tissue having reproducible properties and similar quality can be generated from cells from different donors.

The fusion of a number of cell aggregates (spheroids) enables a much more versatile modelling towards the tissues of different size, such that the functional fusion tissue formed preferably in vitro for example could be adapted ideally to the corresponding size and shape of the respective defect, and special forms, such as meniscus tissue could also be engineered. In order to further improve the tissue functionality and the quality features of the functional fusion tissue, other known methods for stimulation of the differentiation can be used additionally where appropriate. In particular for cartilage tissue, this includes the mechanical stimulation for example via pressure application (compression and decompression) or the cultivation in special bioreactors and spinner flasks in order to thus simulate the natural processes and effective forces in the body, for example when running. This should on the one hand supply the functional fusion tissue optimally with nutrients and on the other hand should also ensure a conduction of external signals (mechanotransduction), which in turn stimulates the synthesis of extracellular matrix components.

Particularly in a society in which the life expectancy of humans is steadily increasing, health is increasingly confronted by degenerative diseases of the joints and of the musculoskeletal system as a whole, for example as a result of arthrotic degradation processes. In clinical practice and in research, there is thus a great need for functional replacement tissues produced in the laboratory. In the previous prior art, the mentioned cell aggregates have become established in this field in the form of spheroids, but are clearly limited in terms of their regeneration capacity and the treatable defect size. The functional fusion tissues now producible with the fusion cultivation constitute an improvement under tissue engineering techniques compared with individual cell aggregates (spheroids) and fusion with just 2 spheroids. With the method according to the invention, it is possible to generate differentiated, shapeable body tissue of different size. The use of the functional fusion tissue for the development of usable tissues for reproduction of body materials of different musculoskeletal origin, for example for use in regenerative medicine and in the pharmaceutical industry, for the testing and improved understanding of disease mechanisms and as a platform for the testing of new drugs is conceivable.

Within the scope of this invention, a model for in vitro chondrogenesis has been created with implications for both fundamental research and clinical approaches. Human chondrocytes are used in order to produce cartilage-like three-dimensional in vitro microtissue with use of the fusion culture according to the invention together with the differentiation-promoting bioactive molecules, but without the aid of any framework material. Following the expansion of isolated chondrocytes in a monolayer culture, accompanied by the dedifferentiation of the cells, adequate chondrogenic stimuli were necessary for redifferentiation. These were provided on the one hand by cultivation of chondrocytes in a 3D environment and on the other hand by addition of growth factors, such as transforming growth factor beta-2 (TGF-β2) and L-ascorbic acid, as antioxidant for prolyl hydroxylase, which is important for faultless collagen synthesis.

Since mesenchymal condensation is one of the earliest steps during the development of many tissues, such as bone, muscles, kidneys or cartilage, the cultivation of human chondrocytes in a 3D environment in vitro in the form of spheroids mimics this aggregation process of mesenchymal cells as the first step of embryonal chondrogenesis. In addition, the combination of a number of individual spheroids with fused tissues as a second aggregation step provides a new method that surprisingly promotes the differentiation of cartilage-like in vitro tissues. In order to evaluate the quality of the produced cartilage-like constructs, differentiation markers were used. Of these markers, collagen type II is the characterising protein for hyaline cartilage, whereas the expression of collagen type I is used to confirm dedifferentiated regions, which are similar to fibrous connective tissue, in the in vitro tissues. In addition, a high content of proteoglycan is important for functional cartilage, and the small intracellular protein S100 is also an indicator for differentiated cartilage cells. Although S100 is used rather seldom as a marker for chondrocytes, it can demonstrate cartilage and chondrocyte differentiation since it is known that a reduced 5100 expression in human articular chondrocytes correlates with cumulative population doubling. The different S100 proteins, for example S100A1 and S100B, are involved in a wide spectrum of intracellular processes, such as cell-cell communication, cell shape, cell structure and cell growth, but also in the intracellular calcium-dependent signal transduction, and behave similarly to cytokines.

The chondrocytes were cultivated for approximately 40 days in a 3D environment, which was partially enriched with differentiation factors. The engineered cartilage-like microtissues were analysed histologically and immunohistochemically in order to determine the quantity and distribution of tissue-specific matrix components. Since the presence of cartilage matrix molecules and key markers at protein level is important for the quality assessment of the produced microtissue, in situ histological and immunohistochemical detection techniques were used. The in vitro chondrogenesis was demonstrated by aged human chondrocytes in the three-dimensional “two-step” fusion culture according to the invention alongside the formation of cartilage-like microtissues without use of any framework or supporting gel material. The fusion tissues according to the invention are a model system for the study both of the metabolism of the cells in question, for example of chondrocytes, and activity thereof in a three-dimensional configuration. In addition, the produced fusion tissues, for example cartilage microtissues, are suitable in an autologous application as transplants, in particular for traumatic defects.

Chondrocytes locked in the cell cycle which have been isolated from cartilage tissue began to again multiply in a monolayer culture. When cultivated in series as monolayer, they started to synthesise cartilage-specific macromolecules and were replaced by molecules that are normally expressed by mesenchymal cells of other connective tissues.

It is known in the prior art to preserve a chondrocyte-specific phenotype in various culture systems. Good results have been obtained by growing human chondrocytes on biologically degradable frameworks (scaffolds). However, most synthetic polymer matrices tend to break down at a considerably acidic pH value, which has proven to be harmful for implanted cells and surrounding tissue. Furthermore, the influence of the framework material on chondrocyte behaviour and cell-cell interactions is often difficult to distinguish. In contrast to these approaches, the method according to the invention and the functional fusion tissues obtained are based on a framework-free induction of cartilage-like in vitro tissues, wherein the mimicry of the first step of in vivo chondrogenesis is utilised. The cells are not forced into a fixed framework structure, which can enable an improved integration into a given articular cartilage defect. In the prior art, only redifferentiation of animal chondrocytes by 3D culture systems is described. The production of three-dimensional cartilage tissue structures from adult human chondrocytes is more difficult, however. In enormous numbers, chondrocytes isolated from healthy joints of animals can be used directly for 3D cultures, whereas in vitro studies with human chondrocytes in most cases require monolayer expansion steps. Chondrocytes of various knee joints of animals already differ in terms of their in vitro biology from cells that have been isolated from the corresponding surface of joints from humans.

The resultant individual spheroids or fusions with use of human chondrocytes constitute a gradual redifferentiation of cells in these microtissues. This redifferentiation process was influenced biochemically by supplementing the medium with TGF-β2. In relation to cartilage, TGF-β is released functionally, and the corresponding receptors are also expressed in chondrocytes. A series of studies demonstrated the role of TGF-β and insulin-like growth factor I (IGF-I) as key mediators in the promotion of tissue repair by increased production of primary components of the articular cartilage matrix. However, the effect of TGF-β on the matrix metabolism in chondrocytes is disputed. Conflicting reports have demonstrated increases and decreases of proteoglycan synthesis, an intensification of the differentiation, and a strong increase or an inhibition of growth. In the present invention, TGF-β2 has proven to be an effective promoter of chondrogenic differentiation in the 3D culture systems.

A combination of TGF-β2 with L-ascorbic acid in the culture medium led to similar results in relation to the chondrogenic redifferentiation in microtissues. With regard to the supplementation with ascorbate, it is often reported that it conveys differentiation-promoting effects in view of the chondrogenic cell line. The effect of ascorbic acid on proteoglycan synthesis by chondrocytes is the subject of controversial debate. The results of the present invention show that ascorbic acid alone could not stimulate PG synthesis. Safranin O positivity was demonstrated in fusions that were cultivated with or without ascorbic acid, which indicates that it is not ascorbate, but the fusion culture according to the invention that acts in a stimulus-inducing manner. It is generally assumed that ascorbate modulates the collagen production as a result of its effect on prolyl hydroxylation. Interestingly, collagen type II was increased neither in individual spheroids nor infusions by addition of L-ascorbic acid alone. By contrast, collagen type I was expressed to a stronger extent in individual spheroids cultivated in ascorbate-containing medium compared with the spheroids cultivated in basal medium. This phenomenon indicates that L-ascorbic acid in particular stimulated collagen synthesis or collagen assembly generally instead of collagen type II, and ascorbate promotes the production of the collagen type for the expression of which the transcription machinery of the cells is programmed. Since expanded, dedifferentiated chondrocytes were used in the method according to the invention in a monolayer culture, it may be that the cells tend towards synthesis of collagen type I, which was in turn favoured by the presence of L-ascorbic acid. The combination with TGF-β2 appeared to reverse this process, since TGF-β2 can selectively trigger the gene expression of collagen type II. The protein expression in individual spheroids and fused microtissues changed towards an improved chondrogenic differentiation, which was demonstrated by an increased expression of collagen type II and S100, which was accompanied by a down-regulation of collagen type I, which was limited to the edges of the microtissue. An additional differentiation-intensifying effect is achieved by the method according to the invention. The fusion cultivation according to the invention improved cartilage tissue formation in any media condition compared with individual spheroids. The effect promoting the differentiation in fusions is cell condensation, which is the key stage during the development of skeletal tissues and facilitates the selective regulation of genes specific for chondrogenesis. Fibronectin and TGF-β are involved in condensation formation, in particular during the initiation phase. TGF-β regulates a series of molecules to this that are associated with pre chondrogenic condensations, including tenascin, fibronectin, N-CAM and N-cadherin. Cell-cell interactions and communication dependent on gap junctions are crucially involved with chondrogenic differentiation. Adult articular cartilage chondrocytes exist as individual cells, which are embedded in the extracellular matrix, and direct intracellular communication takes place via gap junctions predominantly beneath the flattened chondrocytes, which face the outer cartilage layer. Chondrocytes extracted from adult articular cartilage and grown in a primary culture, however, express Connexin 43 (Cx43) and form functional gap junctions which can maintain the propagation of intercellular calcium waves. These mechanisms and components, which are relevant for cell condensation and communication, explain the superior chondro-inductive effect of the fusion culture according to the invention. When starting the fusion process, a number of layers of the surfaces of in vitro tissues were arranged in close contact with one another, whereby cell-cell interactions and the communication conveyed by gap junctions are made possible and associated processes are influenced, such as modified exchange of Ca²⁺ or secondary messenger, which leads to the promotion of cartilage differentiation. In view of the mesenchymal condensation as one of the earliest steps during cartilage development in vivo, the individual spheroids mimic this process in vitro. The combination of a plurality of individual spheroids with fused microtissues as second aggregation step further promotes the mimicry of this development stage of embryonal cartilage formation.

S100 is a marker in human chondrocytes. The conversion of freshly isolated post-mitotic chondrocytes into a monolayer culture leads to a restart of proliferation, accompanied by a reduction and the stop of collagen type II expression in the first passages, whereas S100 protein could be detected after 4 population doublings or even up to more than 22 PDs. Collagen type I was progressively expressed in parallel, which started already in cells of passage 2. The self-aggregation of dedifferentiated, proliferating cells led to a proliferation stop and the subsequent expression of the S100 protein, independently of the media composition and culture condition. The expression of S100 was even higher in the presence of TFG-β and in all microtissues produced with the fusion technique according to the invention. By contrast, antibodies against collagen type II were hardly able to detect this protein in individual spheroids in basal medium and in medium enriched with ascorbate. In order to express collagen type II in microtissues, an extended stimulation of the differentiation process was necessary, i.e. by fusion formation and/or biochemically. The described sequential occurrence of the chondrocyte marker proteins S100 and collagen type II in differentiating microtissues also coincides with the context that S100 proteins are targets of the trios of SOX-transcription factors (SOX9 and coactivators thereof SOX5 and SOX6) and that the transcription factor SOX9 plays key roles in successive steps of the chondrocyte differentiation pathway. The S100 protein is used as an earlier chondro-specific cell marker, which is suitable for quality control of cells in culture and even in in vitro tissues. In addition, S100 is suitable for distinguishing chondrocytes from other mesenchymal cells in connective tissues, such as osteoblasts or fibroblasts, which enables an exclusion of a contamination of the cells in engineered cartilage-like tissue constructs.

The coexpression of collagen type II and S100 at protein level in certain regions of microtissues was clearly visible in individual spheroids and infusions that had been cultivated in TGF-β2-containing medium, and this was confirmed by immunofluorescence. The marked outer edge, positive for collagen type I, was confirmed by means of immunofluorescence in cryosections of individual spheroids and fusions in the presence of TGF-β2. In these regions, collagen type II was practically absent, which represents a small surface layer that mimics fibrous connective tissue and which reflects similarities to the composition of native articular cartilage, which could in turn be confirmed with the aid of an immunohistochemical technique.

The cultivation of expanded and dedifferentiated human articular chondrocytes in the 3D environment according to the invention led to the formation of microtissues in the form of individual spheroids or fusions and results in the redifferentiation of cells in those cartilage-like in vitro tissue constructs. The relevance of S100 as marker for the early chondrocyte differentiation was disclosed by an “expression shift” described for the first time compared with collagen type II, in other words late down-regulation of the S100 expression during the dedifferentiation in a monolayer culture, but early and easier (only by aggregation in the absence of stimulation factors) up-regulation during redifferentiation in a 3D culture before collagen type II was even reexpressed.

The present invention provides data that shows that the method according to the invention (“fusion culture technique”) promotes chondrogenic differentiation in vitro, whereby the arrangement of a self-produced extracellular matrix is introduced, which is composed predominantly of collagen type II and proteoglycans. In individual spheroids, there was only a slight expression of this cartilage marker in basal medium and in the presence of L-ascorbic acid. This limitation of the matrix production could be overcome by the formation of functional fusion tissue. The method according to the invention together with suitable stimulatory growth factors induced synergistically the reexpression of the cartilage phenotype and provided a platform technology for producing framework-free transplants, implants and tissue compositions for humans in vitro that can be applied clinically for example to the regeneration of traumatic cartilage defects, osteoarthritis and rheumatic diseases.

The following figures and examples will explain the invention, but without limiting the invention to the figures and examples.

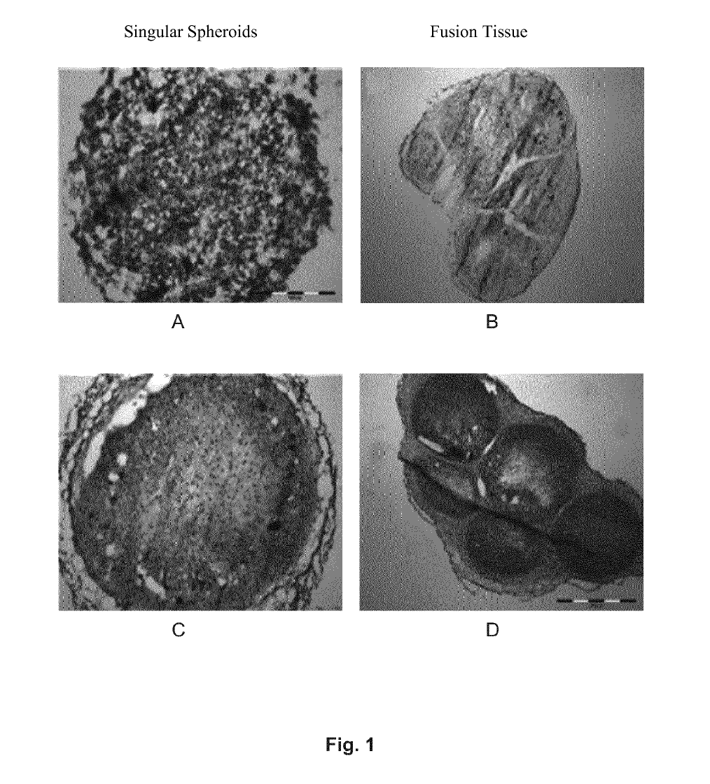

FIG. 1: Illustration of the quality increase by production of fusion tissue from human chondrocytes on the basis of the formation of a cartilage-specific extracellular matrix. Proteoglycans in individual spheroids and fusion tissues characteristic for cartilage tissue were detected by means of Safranin O staining (red=proteoglycans).

A: Individual spheroid in basal medium does not present any proteoglycan synthesis (no red staining). B: Fusion culture of 5 individual spheroids in basal medium induces the production of proteoglycans and incorporation into the extracellular matrix of the fusion tissue (red staining). C: Individual spheroid in basal medium enriched with TGF-β2 promotes proteoglycan formation. D: Fusion tissue in the presence of TGF-β2 stimulates the synthesis and secretion of proteoglycans to a significantly increased extent (intense red staining).

Individual spheroids (A and C) were recorded with a higher size increase; diameter approximately 1000-1300 μm.

Fusion tissues with a smaller size increase were recorded; Size: approximately 2000×3000 μm.

### EXAMPLES

### Example 1

### Cell Source and Monolayer Culture of Human Articular Chondrocytes

Articular cartilage was taken from human femur condyles of patients who had undergone knee surgery. The results were obtained by carrying out three independent tests with cartilage from three different patients. Cartilage tissue was scraped off from the condyles using a sharp scalpel, and chondrocytes were isolated from the surrounding matrix by mechanical size reduction of the tissue using a scalpel, followed by enzymatic treatment. The chopped tissue was then introduced into alpha medium and HAMS F12 (1:1) with collagenase type II (350 E/ml). The closed tube was placed in a shaker with interval mixing at 300 rpm and was incubated for 20 h at 37° C. The extracted chondrocytes were centrifuged at 300×g for 5 min. The supernatant was removed and the pellet was resuspended with 10 ml alpha medium plus HAMS F12, which was enriched with 1% L-glutamine and 10% human serum (serum pool from willing donors) and is referred to hereinafter as basal medium. The chondrocytes were plated in a cell density of 2×10⁴ cells/cm². The cells were expanded in a monolayer culture at 37° C. and 5% CO₂ for two passages.

### Example 2

### Production of Cartilage-Like Microtissues

In order to induce microtissue formation, chondrocytes were sown in wells of 96-well plates coated with agarose in a concentration of 3×10⁵ cells/well in 200 μl basal medium per well. After two days, stable chondrocyte aggregates had already formed, which were then cultivated under different conditions in order to promote the redifferentiation.

### Example 3

### Production of Fusion Tissues and Conditions for Chondrogenic Redifferentiation

Besides the special cultivation conditions, which enable the fusion of individual spheroids with one another, certain bioactive substances were also used in order to intensify the redifferentiation. The induction of fused aggregates was achieved by combining five individual spheroids in a well of a 96-well plate. The individual spheroids and the fusion tissue were cultivated under four different conditions in view of the presence of bioactive molecules. The in vitro aggregates were cultivated either in basal medium, which is also abbreviated hereinafter as BM, in BM supplemented with 50 μg/m1 L-ascorbic acid, in BM plus 5 ng/ml TGF-β2 or in BM supplemented with 50 μg/m1 L-ascorbic acid and 5 ng/ml TGF-β2. The total cultivation time for all microtissue in the aforementioned conditions was six weeks, wherein the medium was replaced three times per week in the case of the individual spheroids and every day in the case of the fused microtissue.

### Example 4

### Analysis of in Vitro Cartilage Tissue

After 6 weeks, the in vitro tissue constructs were harvested and prepared for further analysis. The construct diameter was calculated by means of image analysis from the flat region. This was assessed with an inverse microscope with phase contrast CKX 41, a digital camera DP 71 and the image analysis software Cell^{F}. The tissue constructs were rinsed in PBS, embedded in Neg-50 Frozen Section Medium and cut with use of a cryomicrotiome. The 7 μm cryosections were dried in air and analysed directly or stored at −20° C.

### Example 5

### Histology and Immunohistochemistry

Prior to the analyses, the monolayer chondrocytes from passage 2 and the tissue cryosections were fixed on the glass slides in a two-step method. Firstly, they were fixed with 4% formaldehyde at 4° C. for 10 min. Then, the glass slides were incubated in a 1:1 mixture of methanol and acetone at −20° C. for 10 min. After this fixing process, the slides were rinsed for 3 to 5 min in PBS. A histological staining with haematoxylin and eosin was performed for the morphological analysis of the cells and tissue and with Safranin O Fast Green in order to detect glycosaminoglycans (GAGs). The fixed chondrocytes and cryosections were stained immunohistochemically for human collagen type I, type II and S100. The slides were rinsed with PBS and were incubated for 20 min at room temperature (RT) with goat normal serum that had been diluted 1:50 in PBS/0.1% BSA, in order to block unspecific binding. Primary antibodies were diluted as follows in PBS/0.1% BSA: anti-collagen type I (1:1000), anti-collagen type II (1:1000) and anti-S100 (1:400). The cells and the cryosections were incubated with the primary antibodies overnight at 4° C. in a moisture chamber. The slides were washed three times with PBS and were then incubated for 1 h in the dark at RT in a moisture chamber with Cy3-conjugated goat anti-mouse antibody (collagen type I and II) and goat anti-rabbit antibody (S100) that had been diluted 1:600 in PBS 0.1% BSA with DAPI (1 μg/ml), in order to stain the cell nuclei. The slides were washed three times with PBS and the cells and tissue sections were then mounted in fluorescent mounting medium and covered with a cover glass in order to prevent fluorescent bleaching. Lastly, the slides were stored in the dark at 4° C. until analysis by means of fluorescence microscopy. Cryosections of native human articular cartilage were used as positive control for collagen type II and S100 and as negative control for collagen type I. In addition, all tests included as negative control the replacement of the primary antibody by PBS as a check on unspecific binding of the secondary antibody.

### Example 6

### Phase Contrast Microscopy for Cell Culture Documentation

Photos of the individual spheroids and of the fusions were taken in black and white with the CFX 41 light microscope equipped with the DP 71 camera. The documentation was performed with Cell^{F} image analysis software for microscopy.

### Example 7

### Colour Microscopy for Histological Specimens

The results of the histological analyses were documented with use of the BX 41 microscope, which was equipped with the Color View I camera, and Cell^{D} image analysis software.

### Example 8

### Fluorescence Microscopy for Immunohistochemical Analyses

The fluorescence of the Cy3-conjugated antibodies and of DAPI of the immunohistochemically stained cells and cryosections was made visible using the computer-assisted IX81 fluorescence microscope system with an MT20 xenon burner. The image documentation and evaluation was performed with the F-View II digital camera and Cell^{R} image analysis software for microscopy.

### Example 9

### Summary of the Results

### Example 9.1

### Analysis of Human Articular Cartilage Tissue

Samples of human hyaline cartilage that had been isolated from three different donors were analysed by means of histology and immunohistochemistry. The analyses of a sample are described representatively for all donors hereinafter. The HE staining shows the typical structure of human hyaline cartilage with elongate flattened cells in the surface zone and rounded cells, which are often arranged in small isogenic groups (lacunas) in the middle zone of the tissue. It was found that the chondrocytes (dark blue dots represent the cell nuclei) are separated from the extracellular matrix, which is light blue. The SO Fast Green staining shows the content of proteoglycans in the tissue. Red-stained areas are Safranin O positive (SO positive) and indicate glycosaminoglycans (GAGs). On the whole, the intensity of the SO staining was still rather high, which indicates the presence of proteoglycans in at least 70% of the tissue. Surface regions of the cartilage tissue section, however, were stained green by Fast Green, which implies that the proteoglycans were broken down.

The results of immunohistochemistry showed a predominant expression of collagen type II. The expression of this hyaline cartilage identifier was reduced in particular in the surface regions, which confirms a change of the matrix composition in this zone. S100 proteins were expressed by most of the cells, which could be seen as a positive red staining and which was limited to the cytoplasm of the cells. As expected, collagen type I was not expressed in native hyaline cartilage, with the exception of a very thin layer on the surface. Comparable histological and immunohistochemical results were obtained with the cartilage specimens from two other donors.

### Example 9.2

### Dedifferentiation of the Human Chondrocytes Cultivated as Monolayer

During the cell expansion in the monolayer culture, the chondrocytes dedifferentiated and acquired a fibroblast cell form. Immunohistochemical analyses disclosed that human chondrocytes in passage 2 (p2) of a monolayer culture, that is to say after approximately 4 population doublings (PDs), expressed collagen type II only to a very small extent or did not express it at all. By contrast, the protein S100 was still expressed in all cells as typical dotted staining.

Furthermore, the atypical collagen type I was expressed already by the majority of cells after such a short period in culture.

### Example 9.3

### Production of in Vitro Cartilage Microtissues by Special 3D Culture Systems

Conditions of a three-dimensional cultivation led to the formation of microtissues in the form of individual spheroids or fusion cultures. Generally, stable spheroids were formed independently of the presence of L-ascorbic acid and/or TGF-β2 within two days and were more compact in the following 2-3 weeks of cultivation, but remained constant in terms of size, until they were harvested after 6 weeks. The diameter of the individual spheroids was usually in the range of approximately 800-1400 μm. Apart from size differences, the individual spheroids did not demonstrate any identifiable morphological changes due to different media supplements. By contrast, the presence of TGF-β2 and/or L-ascorbic acid with formation of the fused microtissue appeared to have an influence on the degree of fusion, since in any media composition, with the exception of basal medium, the individual spheroids joined to one another to form a rather compact microtissue, which represented a coherent aggregate. Although the spheroids in the basal medium melted at some surface regions, the fused microtissue forms a rather looser aggregate, in which all individual spheroids remain well defined and distinguishable from one another.

### Example 9.4

### Cell/Matrix Morphology and Proteoglycan Synthesis of the in Vitro Cartilage Microtissue

The HE staining of cryosections of individual spheroids and of the fused microtissue (functional fusion tissue) were analysed. In the case of the basal medium, the cell and matrix distributions in the individual spheroids and the fusions were rather similar, although in the fusions regions with increased

ECM production were visible, which was reflected by a larger distance between the chondrocytes. The addition of L-ascorbic acid alone led to unfavourable weak tissue constructs, which was reflected by cracked cryosections. The fused constructs, however, were again more compact, as could be identified by the increased ECM synthesis. Both media compositions, TGF-β2 alone or in combination with L-ascorbic acid, led to an increased matrix synthesis; in particular the fusions however developed a morphology that was similar to native cartilage. The outer ring is very noticeable, with high matrix content and rather flat cells in the individual spheroids and the fusions in basal medium, which was enriched with TGF-β2 and L-ascorbic acid. The presence of the cytokine TGF-β2 in the culture medium induced an increased synthesis of proteoglycans, independently of the fusion culture technique. The addition of TGF-β2 together with L-ascorbic acid led to the best results in respect of Safranin O positivity. By contrast, the effect of L-ascorbic acid alone rather limited the proteoglycan synthesis in the individual in vitro tissues, since practically no GAGs were detectable under these culture conditions. On the other hand, the cultivation of a number of spheroids as fused microtissue enabled the induction of the proteoglycan synthesis independently of the differentiation factors.

### Example 9.5

### Immunohistochemical Analyses of the in Vitro Cartilage Microtissue

The cytokine TGF-β2 alone or in combination with L-ascorbic acid promoted the redifferentiation of chondrocytes in 3D cultures, which led to an increased collagen type II expression. In particular, the fusion cultivation itself in comparison to individual tissue constructs induced an intensified synthesis of collagen type II, even in the basal medium. The fusion cultivation in combination with TGF-β2 supplementation demonstrated an even further increased collagen type II expression, whereas the supplementation with L-ascorbic acid alone in comparison to the basal medium did not induce any visible up-regulation of collagen type II.

Similarly to the expression of collagen type II, the expression of S100 was also increased only on account of the cultivation of individual spheroids as fused microtissue. Again, the differentiation effects of TGF-β2 alone or in combination with L-ascorbic acid contributed to an up-regulation of S100 expression. It is noteworthy that the S100 expression correlated correctly with the localisation of collagen type II in the sections of the individual spheroids and the fusions, in particular in the presence of TGF-β2 alone or plus L-ascorbic acid. Whereas collagen type II was expressed strongly in the inner part of the microtissue, its expression in the outer zones was very weak. Similar expression patterns were observed for S100, in particular in media composition of TGF-β2+L-ascorbic acid, that is to say strong signals in the centres of the tissue, but weak or even absent signals in the outer rings.

In view of collagen type I as a marker for dedifferentiated cartilage, a reduced expression of this protein is observed in the presence of TGF-β2 and TGF-β2 plus L-ascorbic acid. In both the individual spheroids and the fused microtissues, the collagen type I expression was limited to the outer zone of the in vitro tissue. In addition, it is notable that collagen type II was practically absent in regions in which collagen type I was up-regulated, and vice versa.

### Example 9.5

### Quantitative Analysis of the Quality Determination

9.5.1. Content of Collagen Type II

Collagen type II as one of the most important constituents of the extracellular matrix in articular cartilage tissue is expressed in at least 80 to 95% of the tissue section area of fusions. The percentages are given following computer-assisted evaluation/calculation of the positive immunofluorescence for the protein collagen type II based on the entire tissue section area of the fusion tissue.

By contrast, a collagen type II positivity of at most 10% of the tissue section area was obtained in the publication by Anderer et al., 2002.

9.5.2. Content of Proteoglycans, Quantitative Determination

The proteoglycan content of the fusion tissue was on average 300 μg per mg fusion tissue following quantitative determination. The individual measurements gave values in the range of 170-460 μg proteoglycans per mg fusion tissue.

By contrast, no specifications concerning the quantity of proteoglycans is given in the publication by Anderer et al., 2002 (no quantitative data).

1. A method for producing functional fusion tissue comprising producing spheroids, selecting spheroids having a diameter of at least 800 μm, and fusing at least 5 spheroids having a diameter of at least 800 μm.

2. The method according to claim 1, further comprising isolating cells from tissue of human or animal origin to produce isolated cells and wherein the spheroids are produced from the isolated cells.

3. The method according to claim 2, wherein the isolated cells are multiplied and the spheroids are produced from the multiplied cells.

4. The method according to claim 3, wherein the spheroids are produced from the multiplied cells and the multiplied cell are produced by cultivation of at least 3×10⁵ cells per well of a 96-well plate.

5. The method according to claim 4, wherein the spheroids are produced by the cultivation of the cells for 1 to 3 days.

6. The method according to claim 1, claims, wherein the fusion is performed by joint cultivation of 5 or more spheroids for a period from 3 to 7 weeks.

7. The method according to claim 1, wherein the spheroids are applied to a concave surface for the fusion.

8. The method according to claim 1, wherein the spheroids are fused in the presence of one or more differentiation inducer(s), and wherein the differentiation inducer(s) is/are mechanical, chemical or biochemical differentiation inducers.

9. The method according to claim 2, wherein the isolated cells are isolated from tissue of endodermal, ectodermal or mesodermal origin or organ tissue.

10. The method according to claim 2, wherein the isolated cells comprise chondrocytes.

11. A functional fusion tissue obtainable by a method according to claim 1.

12. A preparation, for example a tissue preparation or pharmaceutical preparation, consisting of or comprising functional fusion tissue according to claim 11 and optionally further additives and auxiliaries.

13. A drug, transplant or implant comprising functional fusion tissue according to claim 11 and optionally further additives and auxiliaries.

14. A functional fusion tissue, preparation, drug, transplant or implant according to claim 11 for specific use for treating rheumatic diseases, cartilage defects, bone defects, in particular traumatic cartilage defects and/or bone defects, lesions, in particular traumatic lesions, in cartilage degeneration, bone degeneration, osteoarthritis, for therapeutic cartilage regeneration and/or bone regeneration in vitro or in vivo.

15. A kit or system, for in vitro or in vivo production of functional fusion tissue comprising: at least 5 spheroids having a diameter of at least 800 μm and optionally further additives and auxiliaries.

16. A method for producing foods comprising the fusion of at least 5 spheroids having a diameter of at least 800 μm.

17. A food obtainable by a method according to claim 16.

18. A test system, for example a test kit, comprising
a) functional fusion tissue, a preparation, a drug, a transplant or an implant according to claim 11,
b) optionally further additives and auxiliaries, and
c) detection means.

19. A method for testing substances to be examined, wherein
a) functional fusion tissue, a preparation, a drug, a transplant or an implant according to claim 11,
b) is brought into contact with one or more substances to be examined,
c) the effect of the substance(s) to be examined on the function fusion tissue, the preparation, the drug, the transplant or the implant is detected.

20. The method according to claim 9 wherein the isolated cells isolated from tissue of endodermal, ectodermal or mesodermal origin are isolated musculoskeletal tissue, skeletal tissue, cartilage, bone, meniscus, epithelial tissue, connective tissue, supporting tissue, muscular tissue, smooth muscle, heart muscle, nerve tissue, functional tissue (parenchyma), or intermediate tissue (interstitium), or, wherein the isolated cells isolated from organ tissue are isolated from s liver, kidney, adrenal cortex, stomach, pancreas, heart, lung, skin, cornea, subcutaneous tissue, intestinal tract, bone marrow, brain, thyroid, spleen, joint, or tendon.
